(19) United States
(12) Patent Application Publication
(10) Pub. No.: US 2019/0133480 A1
(43) Pub. Date: May 9, 2019

(54) DISCRETIZED EMBEDDINGS OF PHYSIOLOGICAL WAVEFORMS

(71) Applicant: KONINKLIJKE PHILIPS N.V., Eindhoven (NL)

(72) Inventors: Asif Rahman, Brookline, MA (US); Bryan Conroy, Garden City South, NY (US)

(21) Appl. No.: 16/178,853

(22) Filed: Nov. 2, 2018

Related U.S. Application Data
(60) Provisional application No. 62/583,128, filed on Nov. 8, 2017.

(51) Int. Cl. A61B 5/0468; A61B 5/00; G06N 5/04; G06N 3/08; G06F 17/16; G06N 3/04

(52) U.S. Cl. CPC A61B 5/0468; A61B 5/7275; G06N 3/0454; G06N 3/084; G06F 17/16; G06N 5/046

(57) ABSTRACT

Techniques described herein relate to training and applying predictive models using discretized physiological sensor data. In various embodiments, a continuous stream of samples measured by a physiological sensor may be discretized into a training sequence of quantized beats. A training sequence of vectors determined based on the training sequence of quantized beats and an embedding matrix may be associated with labels indicative of medical conditions, and applied as input across a neural network to generate corresponding instances of training output. Based on a comparison of each instance of training output with a respective label, the neural network and the embedding matrix may be trained and used to predict medical conditions from unlabeled continuous streams of physiological sensor samples. In some embodiments, the trained embedding matrix may be visualized to identify correlations between medical conditions and physiological signs.

### CROSS-REFERENCE TO RELATED APPLICATION

The present application claims the benefit of U.S. Provisional Application Ser. No. 62/583,128, filed Nov. 8, 2017, which is hereby incorporated by reference in its entirety.

### TECHNICAL FIELD

The present disclosure is directed generally to health care. More particularly, but not exclusively, various methods and apparatus disclosed herein relate to training and applying predictive models using discretized physiological sensor data.

### BACKGROUND

The acquisition of continuous-time physiological signals, such as electrocardiogram (“ECG”) signals, photoplethysmogram (“PPG”) signals, and arterial blood pressure (“ABP”) signals, is becoming more wide-spread as patient monitoring technology evolves to offer cheaper and more mobile sensors. One challenge is extracting meaningful features from these waveforms for various down-stream tasks that assist a clinician in better identifying and managing acute patient conditions. One common approach to this problem is to manually extract a handful of well-known and clinically-validated features from the physiological signals and then train a machine learning model on these features. For example, heart-rate variability has been shown to be highly predictive of many acute conditions.

However, restricting a machine learning algorithm to a small set of manually-defined features is unnecessarily restrictive, especially with the advent of more advanced machine learning techniques (e.g., deep learning), which have been shown to be capable of automatically extracting fine-grained patterns and features from complex and high-dimensional datasets. The trouble with these automated techniques, though, is that they often function as a “black box”-they provide very accurate predictive models but it is often nearly impossible to interpret the extracted features or understand the network's internal logic. This can be problematic for healthcare applications when seeking regulatory approval or soliciting widespread trust of the algorithm within the clinical community.

### SUMMARY

The present disclosure is directed to methods and apparatus for training and applying predictive models using discretized physiological sensor data. For example, in various embodiments, a temporally-continuous stream of samples may be obtained from a physiological sensor such as an electrocardiogram (“ECG”), a photoplethysmogram (“PPG”), and/or an arterial blood pressure (“APB”) sensor. The continuous stream of samples may be “preprocessed,” e.g., by being divided into a training sequence of temporal chunks of the same or different temporal lengths. Labels indicative of health conditions may be associated with each temporal chunk of the training sequence. For example, a temporal chunk of a continuous stream of ECG samples that evidences atrial fibrillation (“AF”) may be labeled as such. In some embodiments, these temporal chunks may be further divided into what will be referred to herein as “beats,” which in some embodiments may be represented as feature vectors. The term “beats” as used herein is not limited to heart beats, nor is it necessarily related to heart beats, although that is the case in some embodiments.

Next, predictive models and embeddings may be learned by applying techniques similar to those often applied in the natural language processing (“NLP”) context. For example, each temporal chunk may be treated like a “sentence,” and the individual beats may be treated as individual “words” of the sentence. In some embodiments, the beats may be quantized such that a sequence of beats associated with a given temporal chunk are embedded into an embedding matrix (which in some cases may be a lookup table), e.g., to determine a corresponding vector. Consequently, a training sequence of vectors may be generated for the training sequence of temporal chunks. The training sequence of vectors may then be applied as input across a machine learning model, such as a recurrent neural network, to generate training output. The training output may be analyzed, e.g., by way of comparison with the aforementioned labels, and the machine learning model and/or the embedding matrix (or “embedding layer”) that precedes the machine learning model may be trained based on the comparison.

Once the machine learning model and/or embedding layer are trained, they can be used for various purposes. For example, learned weights and/or hyperparameters of the machine learning model and/or embedding layer can be fixed. Then, a new (or “live”) continuous stream of samples may be obtained, e.g., from the same type of physiological sensor, and preprocessed as described above. The preprocessed data, which may include an unlabeled sequence of vectors generated based on the live continuous stream of samples, may be applied as input across the trained machine learning model to generate output. The output may be indicative of a prediction of one or more health conditions. For example, in the ECG context, one kind of prediction that may be made using models/embedding layers training with techniques described herein is AF.

In some embodiments, the aforementioned embedding layer may be amenable to interpretation, e.g., to lessen the “black box” appearance of the trained machine learning model. For example, the embedding layer may be decomposed, e.g., using eigenvalue analysis. This analysis can be used in some cases to generate a visualization, e.g., for display on a computer display and/or to be printed, of the learned discretized embeddings. For example, the visualization might show that most of the information learned by the embedding layer is contained in particular dimensions, and the rest of the embedding layer may be sparse. In some cases clusters may become evident in the ranges of highest correlation. This information may enable training of a model with fewer dimensions than the original model. As another example specific to the ECG context, Euclidian distances between bins may reveal how the embedding layer distinguishes normal RR intervals from AF RR intervals.

Techniques described herein may give rise to a variety of technical advantages. For example, the machine learning models described herein strike a balance between conventional manual extraction of clinically relevant and well-understood features with automated feature extraction using deep learning. Additionally, in some embodiments, the continuous streams of samples (e.g., waveforms) may be processed into lower dimensional representations (e.g., quantized beats), which require less data and hence are advantageous in mobile applications wherein network traffic and battery life are important. Furthermore, techniques described herein take advantage of the pseudo-periodic nature of many physiological signals (e.g., ECG, PPG, APB) by, for instance, decomposing the physiological signal into a sequence of quantized beats that evolve over multiple (e.g., cardiac) cycles, and detecting patterns in the quantized beats that can be used to make predictions about clinical conditions. Moreover, techniques described herein leverage NLP techniques to learn from temporal (e.g., inter-beat) patterns in physiological signals.

Generally, in one aspect, a method may include: obtaining a first continuous stream of samples measured by one or more physiological sensors; discretizing the first continuous stream of samples to generate a training sequence of quantized beats; determining a training sequence of vectors corresponding to the training sequence of quantized beats, wherein each vector of the training sequence of vectors is determined based on a respective quantized beat of the training sequence of quantized beats and an embedding matrix; associating a label with each vector of the training sequence of vectors, wherein each label is indicative of a medical condition that is evidenced by samples of the first continuous stream obtained during a time interval associated with the respective vector of the training sequence of vectors; applying the training sequence of vectors as input across a neural network to generate corresponding instances of training output; comparing each instance of training output to the label that is associated with the corresponding vector of the training sequence of vectors; based on the comparing, training the neural network and the embedding matrix; obtaining a second continuous stream of samples from one or more of the physiological sensors; discretizing the second continuous stream of samples to generate a live sequence of quantized beats; determining a live sequence of vectors corresponding to the live sequence of quantized beats, wherein each vector of the live sequence of vectors is determined based on a respective quantized beat and the embedding matrix; applying the live sequence of vectors as input across the neural network to generate corresponding instances of live output; and providing (624), at one or more output devices operably coupled with one or more of the processors, information indicative of the live output.

In various embodiments, discretizing the first continuous stream of samples may includes: organizing the first continuous stream of samples into a first sequence of temporal chunks of samples; and, for each given temporal chunk of samples of the first sequence of temporal chunks of samples: discretizing the given temporal chunk of samples into a quantized beat of the training sequence of quantized beats; and matching the quantized beat to one of a predetermined number of bins. In various embodiments, each bin of the predetermined number of bins may correspond to a predetermined vector of the embedding matrix.

In various embodiments, the first and second continuous streams of samples may include electrocardiogram data. In various embodiments, each quantized beat of the training and live sequences of quantized beats corresponds to an RR interval. In various embodiments, one or both of the first and second continuous streams of samples may be discretized at one or more of the physiological sensors, and one or both of the training sequence of quantized beats and the live sequence of quantized beats may be provided by one or more of the physiological sensors to the one or more processors. In various embodiments, one or both of the first and second continuous streams of samples may be discretized using one or more additional neural networks.

In various embodiments, the neural network may include a recurrent neural network. In various embodiments, the recurrent neural network may include a long short-term memory. In various embodiments, training the neural network may include applying back propagation with stochastic gradient descent. In various embodiments, training the embedding matrix may include determining weights of the embedding matrix.

In various embodiments, the method may further include: applying eigenvalue analysis to the embedding matrix to generate a visualization of the embedding matrix; and rendering the visualization of the embedding matrix on a display device. Systems and non-transitory computer-readable media are also described herein for performing one or more methods described herein.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the disclosure.

FIG. 1 schematically depicts one example of components that may be deployed to implement techniques described herein, in accordance with various embodiments.

FIG. 2 schematically depicts one example of how a continuous stream of samples obtained from a physiological sensor may be processed, in accordance with various embodiments.

FIG. 3 depicts one example of how continuous samples may be quantized or embedded, in accordance with various embodiments.

FIGS. 4A and 4B depict an example of how ECG data may be processed, in accordance with various embodiments.

FIGS. 5A and 5B depict examples of how embedding layers trained with techniques described herein may be visualized for interpretation, in accordance with various embodiments.

FIGS. 6A and 6B depict example methods of practicing selected aspects of the present disclosure.

FIG. 7 depicts an example architecture of a computing system that may be used to implement selected aspects of the present disclosure.

### DETAILED DESCRIPTION

The acquisition of continuous-time physiological signals, such as electrocardiogram (“ECG”) signals, photoplethysmogram (“PPG”) signals, and arterial blood pressure (“ABP”) signals, is becoming more wide-spread as patient monitoring technology evolves to offer cheaper and more mobile sensors. One challenge is extracting meaningful features from these waveforms for various down-stream tasks that assist a clinician in better identifying and managing acute patient conditions. One common approach to this problem is to manually extract a handful of well-known and clinically-validated features from the physiological signals and then train a machine learning model on these features. However, restricting a machine learning algorithm to a small set of manually-defined features is unnecessarily restrictive, especially with the advent of more advanced machine learning techniques (e.g., deep learning), which have been shown to be capable of automatically extracting fine-grained patterns and features from complex and high-dimensional datasets. The trouble with these automated techniques, though, is that they often function as a “black box”-they provide very accurate predictive models but it is often nearly impossible to interpret the extracted features or understand the network's internal logic. In view of the foregoing, various embodiments and implementations of the present disclosure are directed to training and applying predictive models using discretized physiological sensor data

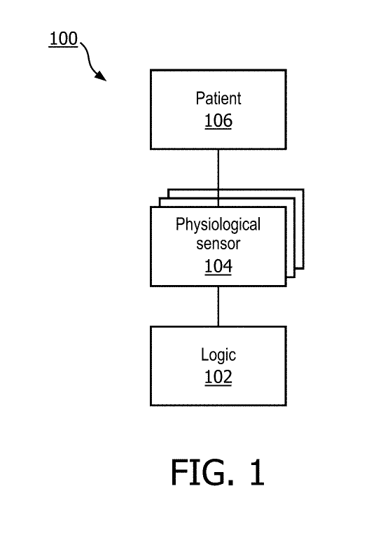

FIG. 1 schematically depicts one example of components that may be deployed to implement techniques described herein, in accordance with various embodiments. In FIG. 1, a logic 102 is operably coupled with one or more physiological sensors 104, which are in turn operably coupled with one or more patients 106. Logic 102 may be operably coupled with the other components using various communication mechanisms, such as one or more busses, wired networking technologies (e.g., Ethernet, USB, serial, etc.), wireless communication technologies (e.g., Z-wave, ZigBee, Bluetooth, Wi-Fi, etc.), and so forth. Logic 102 may take various forms, such as one or more microprocessors that execute instructions stored in memory (not depicted) to perform various aspects of the present disclosure, an application-specific integrated circuit (“ASIC”), a field-programmable gate array (“FPGA”), and so forth. In some embodiments, logic 102 may include one or more microprocessors that are part of a computing system that is connected to the one or more physiological sensors 104.

Physiological sensors 104 may come in various forms to generate various physiological signals. In some embodiments, physiological sensors 104 may include an ECG sensor that produces a continuous ECG signal. Additionally or alternatively, in some embodiments, physiological sensors 104 may include a PPG sensor that produces a PPG signal. Additionally or alternatively, in some embodiments, physiological sensors 104 may include an ABP sensor that produces an ABP signal. In some embodiments, one or more aspects of the preprocessing described below may be performed by the physiological sensor 104 itself, and the preprocessed data may be provided to logic 102. This may conserve memory and/or network bandwidth, which may be important if logic 102 is part of a resource-constrained device such as a mobile phone.

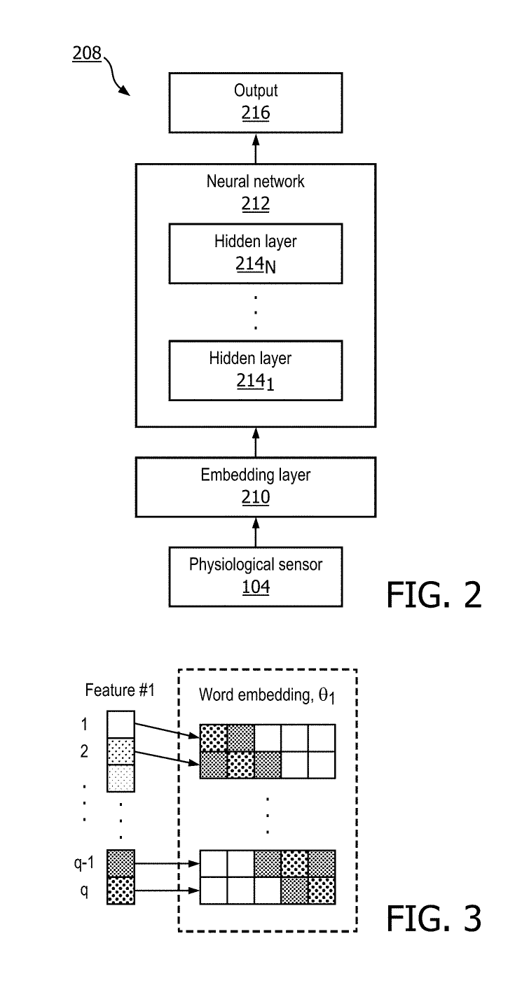

Logic 102 (and in some cases, physiological sensor(s) 104) may be configured to perform various aspects of the present disclosure. FIG. 2 schematically depicts, at a high level, an example 208 of how data may be processed. A continuous stream of samples is acquired at physiological sensor 104. Physiological sensor 104 and/or logic 102 (not depicted in FIG. 2) may preprocess the continuous stream of samples into an embedding layer 210. In some embodiments, the preprocessed data may be a discrete-valued input that is embedded, e.g., at embedding layer 210, into a higher dimensionality space (e.g., matching a quantized beat to a bin that corresponds to a vector of the embedding layer 210). In some embodiments, rows (e.g., vectors) of the embedding layer 210 (or “embedding matrix”) may be applied as input across a neural network 212, which may include one or more hidden layers 214_{1-N}, to generate output 216. Output 216 may be, for instance, an indication or prediction of a health condition.

Now, aspects of the data flow 208 of FIG. 2 will be described in more detail. In some embodiments, logic 102 and/or physiological sensor(s) 104 may be configured to obtain a first continuous stream of samples from one or more physiological sensors 104. This first continuous stream of samples may be obtained from physiological sensor 104 in real time or may be obtained from a log of previously-recorded streams of samples. The latter is particularly true where the first continuous stream of samples is to be used to train one or more embedding layers and/or machine learning models, as will be described herein.

In various embodiments, logic 102 (and in some cases, physiological sensor(s) 104) may be configured to preprocess the first continuous stream of samples to generate a training sequence of quantized beats. In some embodiments, a first step of preprocessing may be to divide the first continuous sequence of samples into temporal chunks, resulting in a sequence of temporal chunks, x₁, x₂, . . . , xₙ. Each temporal chunk x may be represented, for instance, as a feature vector that includes a sequence of samples obtained from the first continuous stream of samples during a particular time interval (e.g., five seconds, ten seconds, thirty seconds, etc.). While each temporal chunk may contain the same number of samples, this is not required.

In some embodiments, a next step of preprocessing may include transforming each temporal chunk of samples into a sequence of what will be referred to herein as “beats.” In various embodiments, each “beat” may be represented by a set of beat-level features (e.g., a feature vector). Mathematically, this new representation can be expressed as a matrix Xᵢ∈ [symbol] ^{b}^{ᵢ}^{×p}, where bᵢ represents the number of beats contained in the ith example, and p is the fixed number of beat-level features extracted. The implementation of this transformation step may depend on both the physical constraints of the physiological sensor 104 and the complexity of the extracted beat-level features. For example, if some of the beat-level extracted features are derived from a neural network, explicit beat segmentation may be required prior to feature extraction. In other applications, however, the set of beat-level extracted features may consist of simple-to-compute features such as RR intervals, which can usually be computed using a peak detection algorithm.

In some embodiments, a last step of preprocessing may be to quantize the beat-level features into a set of bins, i.e., to embed the beat-level features into the embedding layer 210 of FIG. 2. This converts the continuous-valued beat-level matrix Xᵢ into a discrete-valued matrix \hat{X}ᵢ∈ [symbol] ^{q}^{ᵢ}^{×p}. Note that the number of bins q, into which each feature j∈{1, . . . , p} is discretized may be treated as a hyper-parameter that is tuned during the training phase (described below). The total number of bins over all features may also be constrained by limitations on network traffic between physiological sensor 104 and logic 102. For example, given that each input j∈{1, . . . , p} is discretized into qⱼ bins, each beat (row of \hat{X}ᵢ) can be encoded using a total of Σⱼ₌₁^{p}└ log₂(qⱼ)┘ bits. Thus, the number of bins qⱼ may be constrained during training so that the total number of bits required to encode each beat does not exceed some pre-defined limit.

In some embodiments, and in some cases after the continuous stream of samples is preprocessed, logic 102 may associate a label with each quantized beat of the training sequence of quantized beats and/or to each vector of training sequence of vectors (described below). As noted above, each quantized beat of the training sequence is generated from samples of the continuous stream that were obtained during a corresponding time interval (e.g., five seconds, ten seconds, thirty seconds, sixty seconds, etc.)—that is, from a temporal chunk of samples. Each temporal chunk of samples may have a label assigned to it, e.g., by a clinician, that indicates a health condition evidenced by the temporal chunk of samples. For example, a cardiologist may manually label temporal chunks of an ECG signal as, for instance, “normal” or “AF.” These same labels may be associated with the quantized beats that were generated from the corresponding temporal chunks of samples.

Referring primarily to FIG. 2, in various embodiments, once the data is embedded in embedding layer 210 to determine a training sequence of embedding vectors, logic 102 may apply the training sequence of embedding vectors, e.g., one after the other according to their temporal order, as input across neural network 212 to generate training output 216. Neural network 212 may take various forms, such as a recurrent neural network (which in some cases may include an LSTM cell) and/or a convolutional neural network.

For each embedding vector, logic 102 may compare the output 216 to the label associated with the given temporal chunk of samples from which the embedding vector was generated. Based on the comparing, logic 102 may train neural network 212. For example, in some embodiments, logic 102 may employ well-known techniques such as back propagation with stochastic gradient descent to alter weights of the hidden layers 214_{1-N} and, in some cases, weights of the embedding layer 210 as well. Thus, in addition to training both embedding layer 210 and neural network 212 to make predictions about health conditions, embedding layer 210, once trained, may be analyzed on its own (e.g., by being visualized as described below) to identify various information about correlations, etc.

Once neural network 212 and embedding layer 210 are trained, they may be used to predict health conditions based on subsequent unlabeled continuous streams of samples received from physiological sensor 104. For example, a subsequent continuous stream of samples may be acquired at/obtained from physiological sensor 104. The subsequent continuous stream of samples may be preprocessed/embedded into embedding layer 210 in a manner similar to that described above to generate what will be referred to herein as a “live” sequence of embedding vectors.

Logic 102 may then apply the unlabeled embedding vectors as input across the neural network 212 to generate “live” output 216. This “live” output may be indicative of a prediction of a health condition, such as AF. In some embodiments, logic 102 may provide, e.g., at one or more output devices operably coupled with logic 102 (e.g., a display device, a speaker, a printout, etc.), information indicative of the health condition prediction. For example, if AF is detected, logic 102 may cause an alarm to be raised, which in some cases may cause one or more communications (e.g., emails, text messages, pages, etc.) to be transmitted to medical personnel. Additionally or alternatively, a log of a patient's health condition(s) over time may be generated for later analysis by a clinician.

### Training Phase

In the training stage of embedding layer 210 and neural network 212, the model parameters may be tuned based on a set of labeled data. Let (\hat{X}₁, y₁), . . . , (\hat{X}ₙ, yₙ) denote a training dataset in which \hat{X}ᵢ is a discretized beat-level feature matrix as described in the preprocessing section above, and yᵢ is the corresponding label assigned to that sequence. The model used to predict yᵢ from \hat{X}ᵢ may in some embodiments be a deep learning recurrent neural network, e.g., inspired by the use of word embeddings in the natural language processing (“NLP”) domain. Analogous to how a sentence may be decomposed into a set of words arising from a finite vocabulary, so each example \hat{X}ᵢ can be thought of as a “beat sentence,” with each beat also arising from a finite vocabulary due to the discretization process described in the preprocessing section above. The goal may be to learn patterns in the “beat sentence” structure that are predictive of the corresponding label.

The purpose of embedding layer 210 is to take a discrete-valued input and map it into a higher-dimensional continuous space. Mathematically, the embedding is defined by a function ƒ: [symbol] ^{p}→ [symbol] ^{d}. In various embodiments, the input may be a discretized beat represented by a p-dimensional discrete vector, and the output may be a continuous-valued d-dimensional vector which will be fed as input to the subsequent recurrent neural network (e.g., 212 in FIG. 2). Due to the finiteness of the input, the simplest implementation of the function ƒ is as a lookup-table. One possibility is to have each of the qⱼ possible values for feature j map to a distinct real-valued d-dimensional (embedding) vector. The output embedding may be a weighted average of the p (one for each feature) d-dimensional embeddings. This results in a total of O(dΣⱼ₌₁^{p}qⱼ) parameters to be learned. This is demonstrated schematically in FIG. 3. Another possibility is to map each distinct p-dimensional discrete input to its own d-dimensional output vector, which results in a much larger number of parameters to learn.

In various embodiments, the output of embedding layer 210 may be applied as input across a neural network 212 (e.g., a deep recurrent neural network, a long short-term memory (“LSTM”) network, or a convolutional neural network), to learn sequential patterns in the “beat sentences” that are predictive of the corresponding label y. The specifics of the network architecture will vary depending on the application, but in some embodiments, the network may contain one or more stacked recurrent neural network layers (e.g., hidden layers 214_{1-N} in FIG. 2) having outputs that feed to a final layer (e.g., softmax) that produces a single predictive output 216.

In various embodiments, parameters of neural network 212 may be trained in conjunction with the parameters from embedding layer 210 to minimize an objective function that quantifies the difference between the predicted output ŷ and the true label y, e.g., such as binary cross-entropy loss. A variety of different optimization algorithms may be applied to minimize this loss, including but not limited to back-propagation with stochastic gradient descent.

### Execution

After training, in the execution phase, the parameters of embedding layer 210 and neural network 212 that were optimized during the training phase are held fixed. Continuous streams of samples from physiological sensor 104 may be preprocessed as described above and applied as input across embedding layer 210 and neural network 212. The output 216 may include real-time predictions of health conditions. Output 216 may be used to notify clinicians (if necessary) in various ways, such as by raising one or more audio and/or visual alarms, transmitting communications to appropriate computing devices (e.g., by way of text message, alerts, emails, etc.), and/or generating reports, e.g., that document a patient's condition over time.

### Interpretation of the Embedding Layer

As noted above, in various embodiments, the trained embedding layer 210 may be interpreted, e.g., using eigenvalue analysis, to make various determinations. In some embodiments, trained embedding layer 210 may be interpreted to make the overall model less opaque (or “black-box”). In some embodiments, after training, embedding layer 210, or θ, may be decomposed through eigenvalue analysis as follows:

θ=U∧U^{T}

where Λ=diag(λ₁, . . . , λₙ), λ₁, . . . , λₙ is a diagonal matrix of the eigenvalues, and U=[e₁, . . . , eₙ] is a matrix of the eigenvectors.

### Use Case: Predicting Arterial Fibrillation

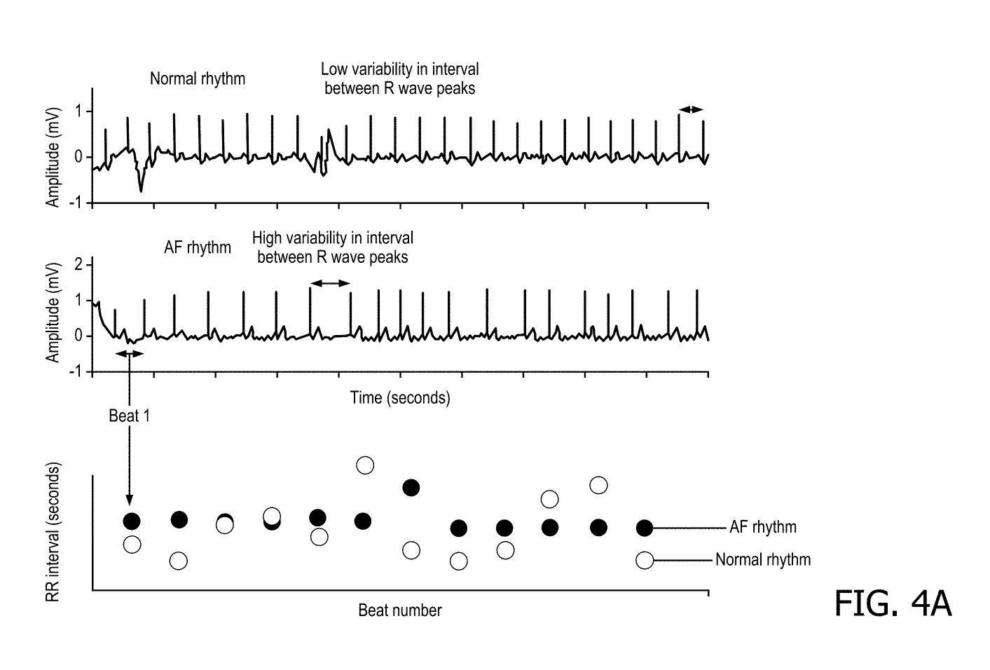

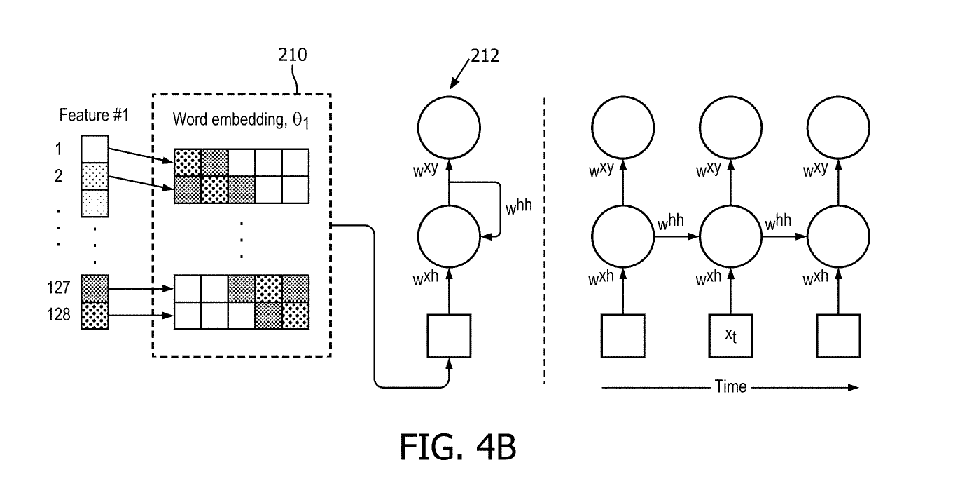

FIGS. 4A and 4B demonstrate one example of how techniques described herein may be used for detection and early prediction of arterial fibrillation (“AF”) from calculated RR intervals, in accordance with various embodiments. AF is characterized by poorly-coordinated atrial activation of the heart and irregular cardiac beating. Most studies related to AF are based on RR (R-wave peak to R-wave peak) interval irregularity from which measures of heart rate variability are calculated. A few studies have implemented features based on the P-wave (which is atrial in origin and is diminished during AF). However, the P-wave is often difficult to pinpoint accurately because original ECG signals may be corrupted with various types of high intensity noise while the P-wave is generally of very low-intensity magnitude. Reliance on P wave features also incurs heavy costs both computationally (since it requires storing ECG and deriving complex features from a continuous-valued ECG signal) and in terms of accuracy. The existing tools for detecting AF in patients are commonly based on measures of heart rate variability. With techniques described herein, by contrast, deep recurrent neural networks typically used with word embeddings are used instead to learn a representation of the dynamics of inter-beat intervals.

At the top of FIG. 4A is an ECG signal that demonstrates low variability in intervals between R-wave peaks, or a “normal” rhythm. The second ECG signal in FIG. 4A demonstrates high variability in intervals between R-wave peaks, which is indicative of a rhythm seen in patients with AF. The chart below the waveforms depicts a discretized representation of the RR intervals depicted in the top two waveforms, with the “normal” rhythm represented by solid black dots and the AF rhythm represented by white dots with black outlines.

In some embodiments, these discretized values representing RR intervals may be used to quantize the waveforms, i.e., to embed representative data into embedding layer 210. In some embodiments, embedding layer 210, which is also referred to as “word embedding, θ₁” in FIG. 4B, may be initialized with random values. However, through the process of training described above, these values may be adjusted to more accurately reflect correlations between various RR intervals. In FIG. 4B, 128 bins are used for quantization, but it should be understood that any number of bins may be employed, and in fact the number of bins may be tuned to satisfy various requirements (e.g., bandwidth usage). In this example, each bin represents a range of RR intervals. Thus, a first RR interval value that falls into a lowest range of potential RR intervals may be mapped to bin 1, a second RR interval value that falls into a second lowest range of potential RR intervals may be mapped to bin 2, and so on.

When a particular RR interval value is mapped to a particular bin, the embedding vector (or row) of embedding matrix 210 that corresponds to the mapped bin may be added to a sequence of embedding vectors. Thus, if ten discretized beats are generated, and each beat includes, as a feature, an RR interval, then ten rows will be generated. These rows are applied as input across neural network 212 (which in this example is a recurrent neural network followed by an LSTM layer). In FIG. 4B, neural network 212 is depicted on the right as being “unfolded” to demonstrate how, over time t, each row (xₜ) is applied as input. Each row/embedding vector will have a corresponding label (e.g., identifying a health condition observed by a clinician). The difference between the output of neural network 212 and the label may be used to train neural network 212 and/or embedding layer 210, e.g., using back propagation and stochastic gradient descent. During the execution phase (i.e., after neural network 212 and embedding layer 210 are already trained), the output of neural network 212 may be a prediction of AF.

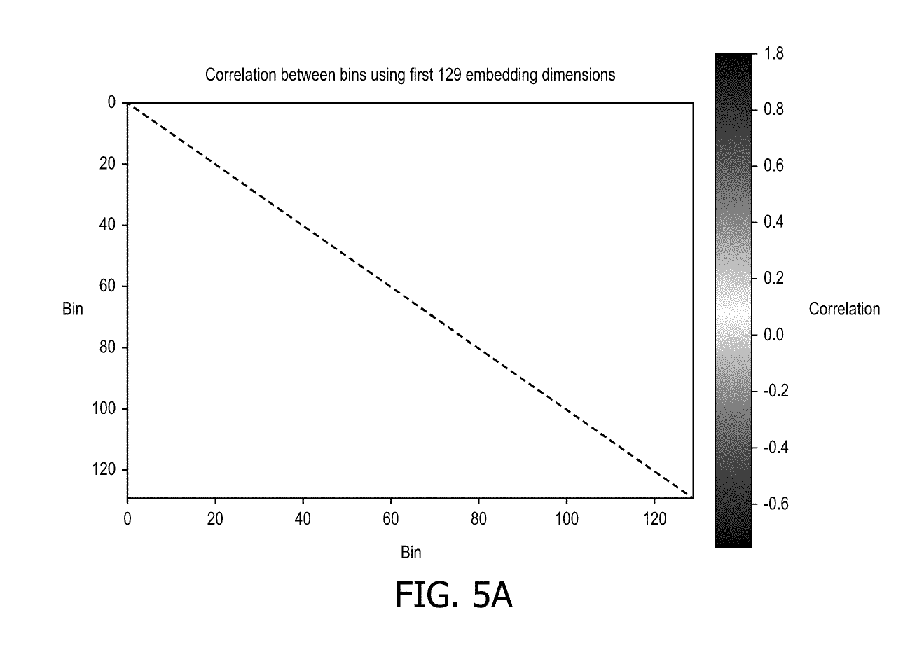

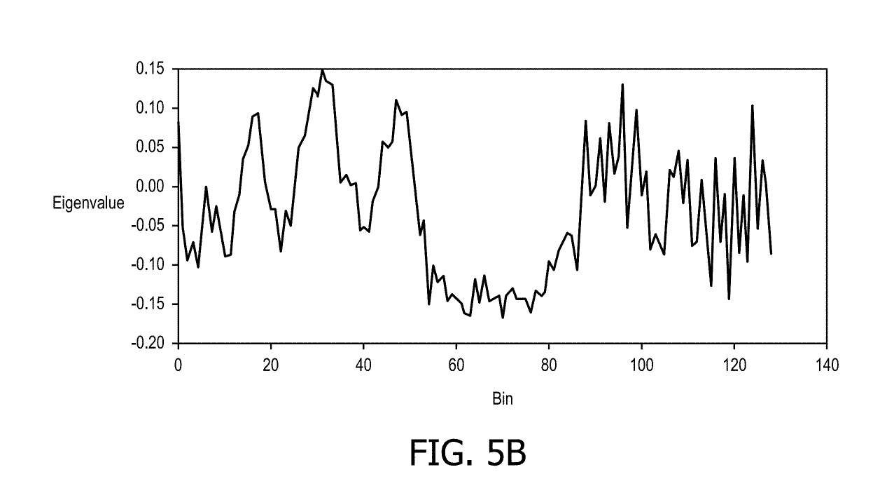

FIGS. 5A and 5B depict examples of visualizations that may be generated based on embedding layer 210 after it is trained in the AF detection scenario. FIG. 5A depicts a visualization (which may be rendered, for instance, on a computer display device) that demonstrates correlation between the 128 bins, or “embedding dimensions,” described above with regard to FIG. 4B. The correlation matrix visualization of FIG. 5A shows that there are several clusters. The largest of these cluster lies in the center and ranges between about bin 50 and about bin 90 (e.g., 770 ms to 1386 ms RR interval) and corresponds to “normal” heart rhythms. Other clusters are found in ranges that can be associated with abnormal heart beats, e.g., AF. Visualizations like that depicted in FIG. 5A can be used by clinicians to provide interpretability to learned embeddings of embedding layer 210. FIG. 5B depicts the first eigenvalue from eigenvalue analysis of the same embedding layer 210 trained for AF detection. Again, this chart shows a cluster around the range of the highest correlations. Numerous other types of visualizations may be generated (e.g., rendered on a screen or on paper) and interpreted in similar fashions.

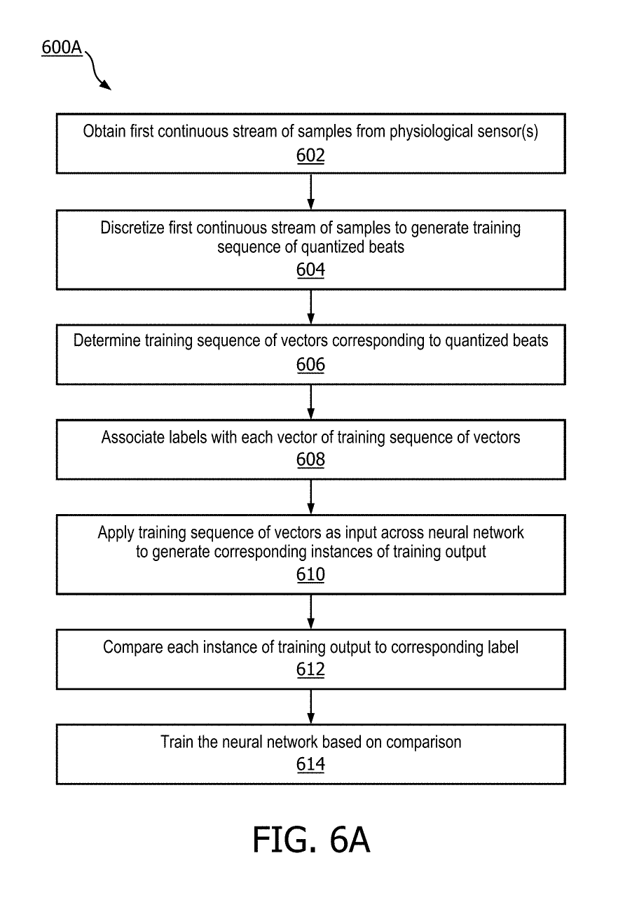

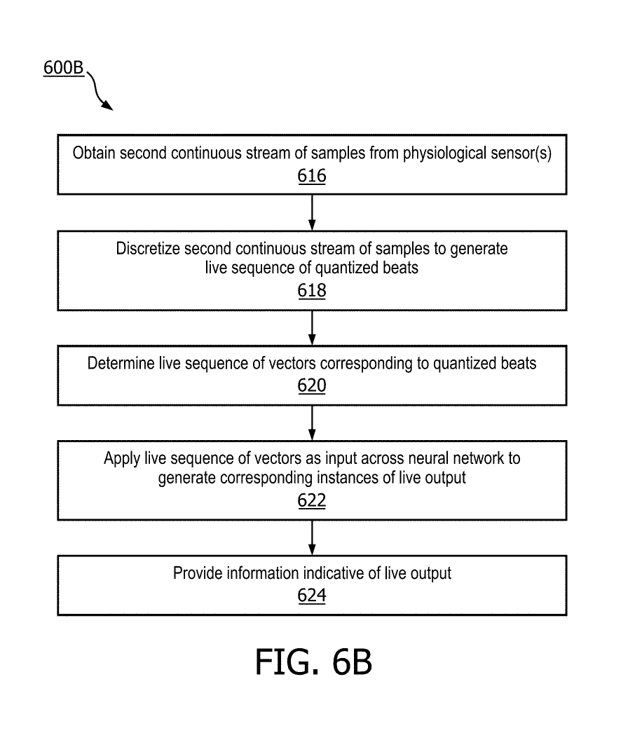

FIG. 6A depicts an example method 600A for training various machine learning models described herein, in accordance with various embodiments. For convenience, the operations of the flow chart are described with reference to a system that performs the operations. This system may include various components of various computer systems, including physiological sensor 104 and/or logic 102. Moreover, while operations of method 600A are shown in a particular order, this is not meant to be limiting. One or more operations may be reordered, omitted or added.

At block 602, the system may obtain a first continuous stream of samples from one or more physiological sensors (e.g., 104). As noted above, various types of physiological sensors 104 may provide such data, such as ECG, PPG, APD, etc. This first continuous stream of samples may be used for training, and thus need not be real time data. More typically, it would be physiological sensor data that is studied by a clinician beforehand. The clinician may label various temporal chunks of the signal with various labels, such as “normal,” “AF,” or with other labels indicative of other health conditions.

At block 604, the system may discretize the first continuous stream of samples to generate a training sequence of quantized beats. As noted above, the preprocessing may include dividing the samples into the aforementioned temporal chunks. At block 606, the system may determine a training sequence of (embedding) vectors that correspond to the training sequence of quantized beats. For example, each quantized beat may be matched to a bin as described above, and then the bin may be used to select a vector (or row) from an embedding matrix (e.g., embedding layer 210). At block 608, the system may associate a label with each vector of the training sequence of vectors. Each label may be indicative of a medical condition that is evidenced by samples of the first continuous stream obtained during the time interval associated with the temporal chunk from which the vector was determined.

At block 610, the system may apply the training sequence of vectors as input across a neural network to generate corresponding instances of training output. At block 612, the system may compare each instance of training output to the label that is associated with the corresponding vector of the training sequence of vectors (i.e., the vector that was applied as input to generate the instance of output). At block 614, based on the comparing, the system may train the neural network, e.g., using back propagation and stochastic gradient descent. In some embodiments, both the neural network and the embedding layer (210 in FIG. 2) may be trained at the same time.

FIG. 6B depicts an example method 600B for applying various machine learning models described herein to obtain predictive output, in accordance with various embodiments. For convenience, the operations of the flow chart are described with reference to a system that performs the operations. This system may include various components of various computer systems, including physiological sensor 104 and/or logic 102. Moreover, while operations of method 600B are shown in a particular order, this is not meant to be limiting. One or more operations may be reordered, omitted or added. Because the operations of method 600B may be performed in many cases after the operations of method 600A, ordinal indicators will pick up from the description of method 600A.

At block 616, the system may obtain a second (e.g., unlabeled) continuous stream of samples from one or more of the physiological sensors. In many cases, the second continuous stream of samples may be a “live” or “real time” stream of samples, though this is not required. At block 618, the system may discretize the second continuous stream of samples to generate a live sequence of quantized beats, as was described above with respect to block 604. At block 620, the system may determine a live sequence of unlabeled vectors corresponding to the live sequence of quantized beats, similar to block 606 of FIG. 6A. At block 622, the system may apply the live sequence of unlabeled vectors as input across the neural network to generate corresponding instances of live output. At block 624, the system may provide, e.g., at one or more output devices associated with one or more computing systems, information indicative of the live output (e.g., a prediction of AF).

In some embodiments, the operations of method 600B may or may not be performed. Instead, the embedding matrix (embedding layer 210) that is trained from method 600A may be used as described to generate visualizations such as those depicted in FIGS. 5A and 5B.

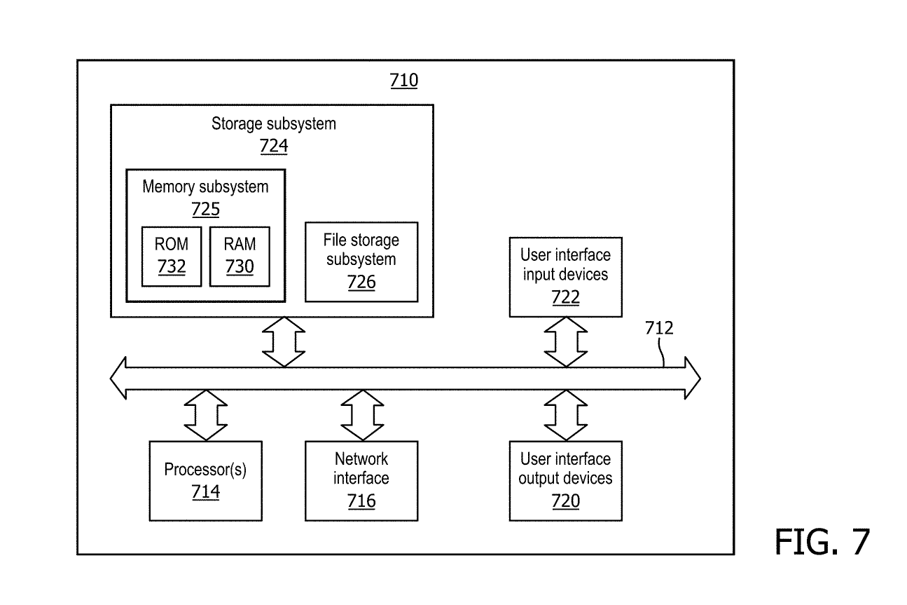

FIG. 7 is a block diagram of an example computing device 710 that may optionally be utilized to perform one or more aspects of techniques described herein. Computing device 710 typically includes at least one processor 714 which communicates with a number of peripheral devices via bus subsystem 712. These peripheral devices may include a storage subsystem 724, including, for example, a memory subsystem 725 and a file storage subsystem 726, user interface output devices 720, user interface input devices 722, and a network interface subsystem 716. The input and output devices allow user interaction with computing device 710. Network interface subsystem 716 provides an interface to outside networks and is coupled to corresponding interface devices in other computing devices.

User interface input devices 722 may include a keyboard, pointing devices such as a mouse, trackball, touchpad, or graphics tablet, a scanner, a touchscreen incorporated into the display, audio input devices such as voice recognition systems, microphones, and/or other types of input devices. In general, use of the term “input device” is intended to include all possible types of devices and ways to input information into computing device 710 or onto a communication network.

User interface output devices 720 may include a display subsystem, a printer, a fax machine, or non-visual displays such as audio output devices. The display subsystem may include a cathode ray tube (CRT), a flat-panel device such as a liquid crystal display (LCD), a projection device, or some other mechanism for creating a visible image. The display subsystem may also provide non-visual display such as via audio output devices. In general, use of the term “output device” is intended to include all possible types of devices and ways to output information from computing device 710 to the user or to another machine or computing device.

Storage subsystem 724 stores programming and data constructs that provide the functionality of some or all of the modules described herein. For example, the storage subsystem 724 may include the logic to perform selected aspects of the method of FIGS. 6A-B, as well as to implement various components depicted in FIGS. 1-2.

These software modules are generally executed by processor 714 alone or in combination with other processors. Memory 725 used in the storage subsystem 724 can include a number of memories including a main random access memory (RAM) 730 for storage of instructions and data during program execution and a read only memory (ROM) 732 in which fixed instructions are stored. A file storage subsystem 726 can provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem 726 in the storage subsystem 724, or in other machines accessible by the processor(s) 714.

Bus subsystem 712 provides a mechanism for letting the various components and subsystems of computing device 710 communicate with each other as intended. Although bus subsystem 712 is shown schematically as a single bus, alternative implementations of the bus subsystem may use multiple busses.

Computing device 710 can be of varying types including a workstation, server, computing cluster, blade server, server farm, or any other data processing system or computing device. Due to the ever-changing nature of computers and networks, the description of computing device 710 depicted in FIG. 7 is intended only as a specific example for purposes of illustrating some implementations. Many other configurations of computing device 710 are possible having more or fewer components than the computing device depicted in FIG. 7.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be understood that certain expressions and reference signs used in the claims pursuant to Rule 6.2(b) of the Patent Cooperation Treaty (“PCT”) do not limit the scope.

What is claimed is:

1. A method implemented at least in part by one or more processors, comprising:
obtaining a first continuous stream of samples measured by one or more physiological sensors;
discretizing the first continuous stream of samples to generate a training sequence of quantized beats;
determining a training sequence of vectors corresponding to the training sequence of quantized beats, wherein each vector of the training sequence of vectors is determined based on a respective quantized beat of the training sequence of quantized beats and an embedding matrix;
associating a label with each vector of the training sequence of vectors, wherein each label is indicative of a medical condition that is evidenced by samples of the first continuous stream of samples obtained during a time interval associated with the respective vector of the training sequence of vectors;
applying the training sequence of vectors as input across a neural network to generate corresponding instances of training output;
comparing each instance of training output to the label that is associated with the corresponding vector of the training sequence of vectors;
based on the comparing, training the neural network and the embedding matrix;
obtaining a second continuous stream of samples from one or more of the physiological sensors;
discretizing the second continuous stream of samples to generate a live sequence of quantized beats;
determining a live sequence of vectors corresponding to the live sequence of quantized beats, wherein each vector of the live sequence of vectors is determined based on a respective quantized beat and the embedding matrix;
applying the live sequence of vectors as input across the neural network to generate corresponding instances of live output; and
providing, at one or more output devices operably coupled with one or more of the processors, information indicative of the live output.

2. The method of claim 1, wherein discretizing the first continuous stream of samples includes:
organizing the first continuous stream of samples into a first sequence of temporal chunks of samples;
for each given temporal chunk of samples of the first sequence of temporal chunks of samples:
discretizing the given temporal chunk of samples into a quantized beat of the training sequence of quantized beats; and
matching the quantized beat to one of a predetermined number of bins;
wherein each bin of the predetermined number of bins corresponds to a predetermined vector of the embedding matrix.

3. The method of claim 1, wherein the first and second continuous streams of samples comprises electrocardiogram data.

4. The method of claim 3, wherein each quantized beat of the training and live sequences of quantized beats corresponds to an RR interval.

5. The method of claim 1, wherein one or both of the first and second continuous streams of samples are discretized at one or more of the physiological sensors, and one or both of the training sequence of quantized beats and the live sequence of quantized beats are provided by one or more of the physiological sensors to the one or more processors.

6. The method of claim 1, wherein one or both of the first and second continuous streams of samples are discretized using one or more additional neural networks.

7. The method of claim 1, wherein the neural network comprises a recurrent neural network.

8. The method of claim 7, wherein the recurrent neural network comprises a long short-term memory.

9. The method of claim 1, wherein training the neural network includes applying back propagation with stochastic gradient descent.

10. The method of claim 1, wherein training the embedding matrix includes determining weights of the embedding matrix.

11. The method of claim 1, further comprising:
applying eigenvalue analysis to the embedding matrix to generate a visualization of the embedding matrix; and
rendering the visualization of the embedding matrix on a display device.

12. A system comprising one or more processors and memory operably coupled with the one or more processors, wherein the memory stores instructions that, in response to execution of the instructions by one or more processors, cause the one or more processors to perform the following operations:
obtaining a first continuous stream of samples measured by one or more physiological sensors;
discretizing the first continuous stream of samples to generate a training sequence of quantized beats;
determining a training sequence of vectors corresponding to the training sequence of quantized beats, wherein each vector of the training sequence of vectors is determined based on a respective quantized beat of the training sequence of quantized beats and an embedding matrix;
associating a label with each vector of the training sequence of vectors, wherein each label is indicative of a medical condition that is evidenced by samples of the first continuous stream of samples obtained during a time interval associated with the respective vector of the training sequence of vectors;
applying the training sequence of vectors as input across a neural network to generate corresponding instances of training output;
comparing each instance of training output to the label that is associated with the corresponding vector of the training sequence of vectors;
based on the comparing, training the neural network and the embedding matrix;
obtaining a second continuous stream of samples from one or more of the physiological sensors;
discretizing the second continuous stream of samples to generate a live sequence of quantized beats;
determining a live sequence of vectors corresponding to the live sequence of quantized beats, wherein each vector of the live sequence of vectors is determined based on a respective quantized beat and the embedding matrix;
applying the live sequence of vectors as input across the neural network to generate corresponding instances of live output; and
providing, at one or more output devices operably coupled with one or more of the processors, information indicative of the live output.

13. The system of claim 12, wherein discretizing the first continuous stream of samples includes:
organizing the first continuous stream of samples into a first sequence of temporal chunks of samples;
for each given temporal chunk of samples of the first sequence of temporal chunks of samples:
discretizing the given temporal chunk of samples into a quantized beat of the training sequence of quantized beats; and
matching the quantized beat to one of a predetermined number of bins;
wherein each bin of the predetermined number of bins corresponds to a predetermined vector of the embedding matrix.

14. The system of claim 12, wherein the first and second continuous streams of samples comprises electrocardiogram data.

15. The system of claim 14, wherein each quantized beat of the training and live sequences of quantized beats corresponds to an RR interval.

16. The system of claim 12, wherein one or both of the first and second continuous streams of samples are discretized at one or more of the physiological sensors, and one or both of the training sequence of quantized beats and the live sequence of quantized beats are provided by one or more of the physiological sensors to the one or more processors.

17. The system of claim 12, wherein one or both of the first and second continuous streams of samples are discretized using one or more additional neural networks.

18. The system of claim 12, wherein the neural network comprises a recurrent neural network.

19. The system of claim 12, wherein training the embedding matrix includes determining weights of the embedding matrix.

20. At least one non-transitory computer-readable medium comprising instructions that, in response to execution of the instructions by one or more processors, cause the one or more processors to perform the following operations:
obtaining a first continuous stream of samples measured by one or more physiological sensors;
discretizing the first continuous stream of samples to generate a training sequence of quantized beats;
determining a training sequence of vectors corresponding to the training sequence of quantized beats, wherein each vector of the training sequence of vectors is determined based on a respective quantized beat of the training sequence of quantized beats and an embedding matrix;
associating a label with each vector of the training sequence of vectors, wherein each label is indicative of a medical condition that is evidenced by samples of the first continuous stream of samples obtained during a time interval associated with the respective vector of the training sequence of vectors;
applying the training sequence of vectors as input across a neural network to generate corresponding instances of training output;
comparing each instance of training output to the label that is associated with the corresponding vector of the training sequence of vectors;
based on the comparing, training the neural network and the embedding matrix;
obtaining a second continuous stream of samples from one or more of the physiological sensors;
discretizing the second continuous stream of samples to generate a live sequence of quantized beats;
determining a live sequence of vectors corresponding to the live sequence of quantized beats, wherein each vector of the live sequence of vectors is determined based on a respective quantized beat and the embedding matrix;
applying the live sequence of vectors as input across the neural network to generate corresponding instances of live output; and
providing, at one or more output devices operably coupled with one or more of the processors, information indicative of the live output.
